(19) United States
(12) Patent Application Publication
(10) Pub. No.: US 2018/0161092 A1
(43) Pub. Date: Jun. 14, 2018

(54) DOUBLE SHAPE CATHETER

(71) Applicant: CathRx Ltd, Homebush Bay, NSW (AU)

(72) Inventors: Roman Greifeneder, Bexley (AU); David Ogle, Cowan (AU)

(21) Appl. No.: 15/580,888

(22) PCT Filed: May 6, 2016
(86) PCT No.: PCT/AU2016/050331

§ 371 Date: Dec. 8, 2017

(30) Foreign Application Priority Data
Jun. 10, 2015 (AU) 2015902173

(51) Int. Cl. A61B 18/14

(52) U.S. Cl. CPC A61B 18/1492; A61B 2018/00791; A61B 2018/1467; A61B 2018/1407

(57) ABSTRACT

A double loop catheter for delivering ablation energy, the catheter comprising a catheter sheath having a proximal end, a distal end and at least one lumen extending therethrough and having a first loop structure having a proximal end and a distal end, and a second loop structure having a proximal end and a distal end. The first loop structure and the second loop structure are receivable in the at least one lumen of the catheter sheath, and the first loop structure and the second loop structure are configurable to extend distally of the distal end of the catheter sheath. The first loop structure comprises a first electrode near to the distal end of the first loop structure and the second loop structure comprises a second electrode near to the distal end of the second loop structure, wherein the first loop structure and the second loop structure are displaceable relative to each other.

### CROSS-REFERENCE TO RELATED APPLICATIONS

This application is a national phase entry under 35 U.S.C. § 371 of International Patent Application PCT/AU2016/050331, filed May 6, 2016, designating the United States of America and published in English as International Patent Publication WO 2016/197186 A1 on Dec. 15, 2016, which claims the benefit under Article 8 of the Patent Cooperation Treaty to Australian Patent Application Serial No. 2015902173, filed Jun. 10, 2015.

### TECHNICAL FIELD

The present disclosure relates to a double shape catheter. More particularly, the present invention may relate to an adjustable double loop catheter with at least one electrode.

### BACKGROUND

Current double loop catheters generally comprise an array of electrodes for diagnostic purposes or an array of ablation electrodes for ablating target tissue of a patient. These double loop catheters are generally positioned by wedging a loop or hook into a target location and performing ablation of target tissue. However, these double loop catheters require that the sensing electrodes generally be turned off while ablating as the energization of the ablation electrodes interferes with the ability of the sensing electrodes to effectively sense. As such, an operator of the device is unable to both effectively sense and ablate at the same time.

Double loop catheters also typically have a shared or common conductive arrangement such that the sensing electrodes and the ablation electrodes receive power through the same conductive elements. This may cause the sensing electrodes to return interfered or incorrectly sensed data.

Other known double loop catheters can have a first and a second loop structure that are substantially radially offset from the axis of the catheter lead or disposed near to the edge of the loop structure. This arrangement may increase the difficulty for the operator to correctly position the catheter in a desired target tissue location. This arrangement may also make it more difficult to correctly position the ablation loop for effective ablation, which may cause the operator to incorrectly burn surrounding non-target tissue. In addition, having a radially offset loop from the catheter may cause misaligned ablation to occur such that one portion of a patient's tissue is energized more than another portion, causing an overburn or scorching of patient tissue causing increasing the time it takes for a patient to recover.

Presently known variable double loop catheters may allow the size or diameter of the loops to be altered. However, these devices generally do not allow for adjustment of size of only a single loop structure, which may cause difficulty in positioning the catheter at a target location in a patient. Other variable catheters have a first loop with a second loop extending from a distal end of the first loop. One disadvantage with this previous catheter configuration is that as the size of one loop is adjusted, the size of the other loop must also be adjusted to ensure that the first loop is concentric to the second loop in use, thereby preventing independent variation of the loops. Further, adjusting the size of one loop may restrict manipulation of the other loop.

Another known catheter device comprises a forward hook and a loop shape. The hook of the catheter is adapted to secure the loop in a desired location in vivo. However, these devices do not provide a sufficient securing for the loop as the hook cannot form a sufficient abutting arrangement or securing point to maintain a desired placement of the catheter for extended periods of time.

Any discussion of the prior art throughout the specification should in no way be considered as an admission that such prior art is widely known or forms part of common general knowledge in the field.

### BRIEF SUMMARY

### Problems to be Solved

It may be an advantage to have a double loop catheter that may sense and irrigate a target location.

It may be an advantage to have a catheter with independent sensing electrodes and energizing electrodes.

It may be an advantage to have a first loop structure and a second loop structure that are axially and/or radially displaceable relative to each other.

It may be an advantage to have a double loop catheter in which the first loop and second loop structures are generally concentrically disposed relative to a catheter sheath.

It may be an advantage to have a double loop catheter in which an operator may selectively alter the size of one loop without altering the size of the other loop.

It may be advantageous to have a double loop catheter with a relatively small French (Fr) diameter.

It may be advantageous to have a catheter that can direct irrigation fluid to a target site effectively.

It is an object of the present invention to overcome or ameliorate at least one of the disadvantages of the prior art, or to provide a useful alternative.

### Means for Solving the Problem

A first aspect of the present disclosure may relate to a double loop catheter for delivering ablation energy, the catheter may comprise: a catheter sheath having a proximal end, a distal end and at least one lumen extending therethrough; a first loop structure having a proximal end and a distal end and a second loop structure having a proximal end and a distal end. The first loop structure and the second loop structure may be receivable in the at least one lumen of the catheter sheath and may be configurable to extend distally of the distal end of the catheter sheath. The first loop structure may comprise a first electrode near to the distal end of the first loop structure and the second loop structure comprising a second electrode near to the distal end of the second loop structure and wherein the first loop structure and the second loop structure may be displaceable relative to each other.

In at least one embodiment, at least one of the first loop structure and the second loop structure may be axially displaceable. The distal end of the catheter sheath may restrict fluid from entering the at least one lumen of the catheter sheath. The first loop structure and the second loop structure may be configured to align about a concentric axis. The second loop structure may comprise at least one fluid aperture. The second loop structure may comprise a plurality of fluid apertures. At least one fluid aperture may be disposed either side of each ablation electrode. At least one of the first loop structure and the second loop structure may comprise a shape-imparting element adapted to impart a shape to the distal end of the respective loop structure. The shape-imparting element may be a stylet adapted to impart a non-rectilinear shape to the distal end of the respective loop structure. The double loop catheter may further comprise a manipulation means such that actuation of the manipulation means may alter a relative size of the loop of at least one of the first loop structure and the second loop structure. Each electrode may comprise at least one fluid aperture adapted to expel a fluid.

Another aspect of the present invention may relate to a catheter for delivering energy to a target location, wherein the catheter may comprise: a catheter sheath having a proximal end and a distal end; a first structure having a proximal end and a distal end and a second structure having a proximal end and a distal end. Each of the distal ends of the first and second structures having a shape imparted thereto. Each of the first structure and the second structure may be adapted to extend from the distal end of the catheter sheath; and wherein the first structure and the second structure may be displaceable relative to each other.

In at least one embodiment, at least one of the first and the second structures may comprise an electrode near to its respective distal end. The electrode may be at least one of a sensing electrode and an energizing electrode. The shape imparted to the first and second structures may be a non-rectilinear shape. At least one of the first structure and the second structure may be telescopically displaceable. The second structure may comprise at least one fluid aperture adapted for expelling a fluid. At least one of the first structure and the second structure may comprise a shape-imparting element.

The catheter may further comprise a manipulation means for manipulating a respective shape-imparting element. The shape of the first structure may be configured to be concentrically aligned relative to the shape of the second structure.

A further aspect of the present invention may relate to a catheter for delivering energy to a target location, wherein the catheter may comprise: a catheter sheath having a proximal end and a distal end and a first structure having a proximal end and a distal end and a second structure having a proximal end and a distal end. Each of the distal ends of the first and second structures may have a shape imparted thereto. The second structure may further comprise a fluid conduit disposed on at least a portion of an outer perimeter of the shape imparted thereto. Each of the first structure and the second structure may be adapted to extend from the distal end of the catheter sheath; and wherein the first structure and the second structure may be displaceable relative to each other.

In the context of the present disclosure, the words “comprise,” “comprising,” and the like, are to be construed in their inclusive, as opposed to their exclusive, sense, that is, in the sense of “including, but not limited to.”

### BRIEF DESCRIPTION OF THE FIGURES

FIG. 1 illustrates a perspective view of an embodiment of a double shape catheter with a first shape structure and a second loop structure;

FIG. 2 illustrates a cut-away view of an embodiment of a portion of a second shape structure for the catheter of FIG. 1;

FIG. 3 illustrates a cross-sectional view of an embodiment of a catheter sheath of the present disclosure;

FIG. 4 illustrates a side view of an embodiment of the catheter of the present disclosure;

FIG. 5 illustrates a side view of a portion of an embodiment of an adjustable double loop catheter in which the first structure is in a retracted position relative to the second loop;

FIG. 6 illustrates a side view of a portion of the catheter of FIG. 5, in which the first structure is in an extended position relative to the second structure;

FIG. 7 illustrates a top view of an embodiment of a double shape catheter in which the first shape structure and the second shape structure are axially displaced relative to each other;

FIG. 8 illustrates a top view of the catheter of FIG. 7 in which the first shape structure and the second shape structure are axially concentric relative to each other;

FIG. 9 illustrates a side view of another embodiment of a double shape catheter wherein the second shape structure comprises a lumen for the first shape structure to be received;

FIG. 10 illustrates a side view of a further embodiment of a catheter with fluid apertures formed adjacent to the electrodes; and

FIG. 11 illustrates an enlarged view of the electrode with fluid apertures as seen in FIG. 10.

### DETAILED DESCRIPTION

Preferred embodiments of the present disclosure will now be described with reference to the accompanying drawings and non-limiting examples.

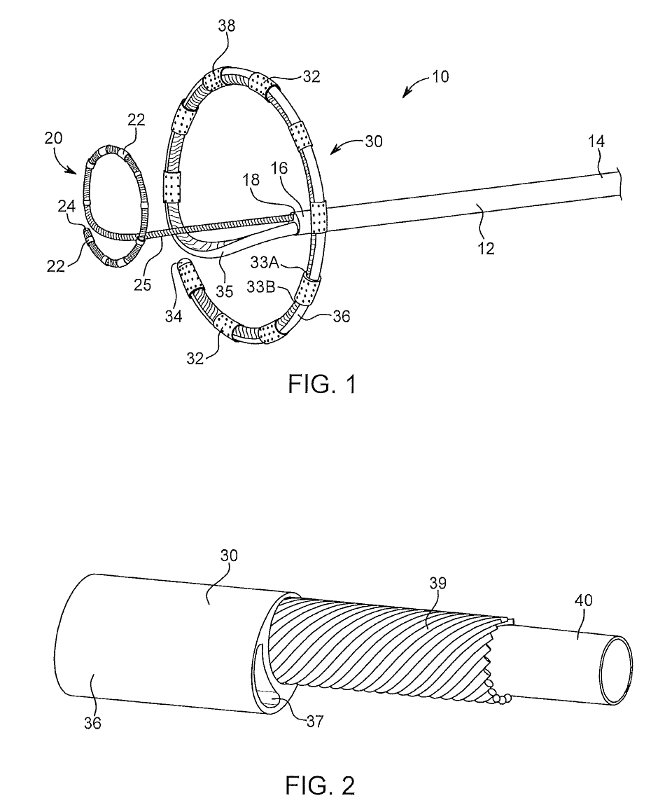

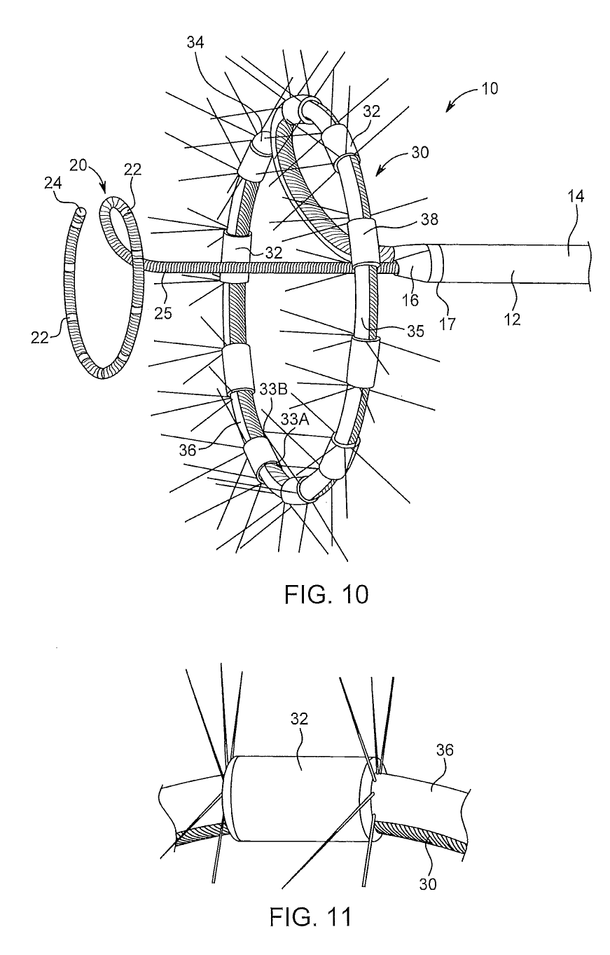

Referring to FIG. 1, there is illustrated an embodiment of the catheter 10 of the present disclosure. The catheter 10 comprises a catheter handle (not shown), a catheter sheath 12 with a proximal end 14, a distal end 16 and at least one lumen extending therethrough. The distal end 16 may have a cap 17 or strain relief 17 (see FIGS. 4 and 10), for example. The distal end 16 of the catheter sheath 12 has at least one opening to allow at least one structure to extend through the lumen and distal to the distal end of the sheath 12. The at least one structure is preferably a loop structure as shown in FIG. 1, however, other shapes may also be used. More preferably, the catheter 10 comprises two structures, a first shape structure 20 and a second shape structure 30, which are configurable such that they may extend distally of the distal end 16 of the catheter sheath 12, as shown in FIG. 1.

As illustrated in FIG. 1, the first shape structure 20 and the second shape structure 30 are independent structures. Having independent structures may allow an operator or clinician to independently adjust or manipulate the relative shape or the relative size of the first structure 20 and/or the second structure 30. In another embodiment, a clinician may adjust or manipulate the deflection of at least one structure 20, 30 such that the catheter 10 can be maneuvered through tortuous anatomy to a target location. Preferably, any manipulation of the first structure 20 and/or the second structure 30 may be effected by at least one control mechanism (not shown) on or near to the catheter handle (not shown).

The first structure 20 comprises a proximal end (not shown) and a distal end 24. Preferably, the distal end 24 of the first structure 20 is free. At least one electrode 22, such as a ring electrode or the like, may be disposed near to the distal end 24 of the first shape structure 20. Preferably, the first shape structure 20 comprises an array of electrodes 22 or a plurality of spaced apart electrodes 22 adapted to sense a target tissue location or electrical signals. In yet another embodiment, an electrode 22 may be adapted to detect or monitor temperature. The first shape structure 20 may be formed with a predetermined shape, such as a linear, non-rectilinear or loop shape. Optionally, an operator or clinician may alter the shape of the first shape structure 20. For example, the first shape structure 20 may be straightened, deflected or otherwise alter shape such that it may be adapted to trek through tortuous anatomy or be adapted for a superior abutting relationship with a tissue of a patient.

In at least one embodiment, the first structure 20 may be radially expanded or contracted by a manipulation means (not shown). This may allow the first structure 20 to form an abutting relationship with a tissue of a patient such that the location of the second shape structure 30 may be releasably fixed in a desired location.

The second shape structure 30 comprises a proximal end (not shown) and a distal end 34. Preferably, the distal end 34 of the second shape structure 30 is free. At least one electrode 32 is disposed near to the distal end 34 of the second shape structure 30. The at least one electrode 32 is preferably an ablation or energizing electrode sufficient to deliver ablation energy to a target tissue. In a further embodiment, the second shape structure 30 comprises an array of electrodes 32 or a plurality of spaced apart electrodes 32. The shape of the second shape structure 30 extends from the distal end of the sheath 16 via bridging portion 35. The bridging portion 35 is formed unitarily with the structure 30. Preferably, at least one electrode 32 may be adapted to sense or monitor temperature.

The second shape structure 30 may be configured to deliver energy, such as RF energy, to a target tissue. The RF energy may be conducted by irrigation fluid, which may be discharged through at least one fluid aperture 38. Alternatively, the second shape structure 30 may be devoid of any fluid apertures 38 and the at least one electrode 32 may provide RF energy directly to or proximal to a target tissue without any irrigation fluid. Optionally, the clinician may use the second structure to burn a grid of rings or shapes at a target site while the first structure is secured in a single location. This may be achieved by displacing the first structure 20 relative to the second structure 30. Forming an ablation grid may reduce the amount of burning that a clinician may need to perform.

A shape-imparting element (not shown), such as a stylet or wire pull system, may be used to impart a predetermined or desired shape to at least one of the first shape structure 20 and the second shape structure 30. Each shape structure may comprise a separate shape-imparting element such that each shape structure may be individually adjusted or altered. For example, a clinician may alter the shape or the size of a shape of the distal end of at least one of the first shape structure 20 and the second shape structure 30. Alternatively, at least one of the first and second shape structures 20, 30 may be formed with a predetermined shape.

Preferably, the first structure and the second structure may be releasably locked or secured in a relative position, such that when the first structure 20 and the second structure 30 are in a desired location a clinician may retain the relative locations of each structure. This may improve the accuracy of the operation and reduce overburn or damage to non-target tissue.

In a further embodiment, the first shape structure 20 can be withdrawn from the catheter sheath 12 or withdrawn at least partially into the catheter sheath 12 such that the second shape structure 30 can be used as an introducer. In an alternative embodiment, the first shape structure 20 can be used as the introducer and the second shape structure 30 may be withdrawn from or partially withdrawn into the catheter sheath 12. It will be appreciated that both the first shape structure 20 and the second loop structure 30 may be configured to be completely withdrawn or at least partially withdrawn from the catheter 10. This may allow the catheter 10 or at least one part of the catheter 10 to be reprocessed for further use.

Preferably, the distal end 16 of the catheter sheath 12 comprises at least one ingress seal (not shown), such as a membrane, which prevents or restricts the flow of fluid into the catheter sheath 12 when in use, but may allow a shape structure 20, 30 to pass therethrough. This may allow insertion or withdrawal of at least one shape structure 20, 30 such that the catheter 10 may be more easily maneuvered to a target location.

The at least one sensing electrode 22 of the first shape structure 20 may be adapted to sense a target location while the second shape structure 30 may irrigate and/or ablate the target area of a patient. Having a first and second shape structure 20, 30 with independent conductors or conductive wiring 39 may allow the catheter 10 to sense and ablate simultaneously such that the operator of the catheter 10 does not have to cease ablating tissue to sense electrical signals near to the target area. This may allow an operator to more effectively ablate a target area and may reduce the procedure time. Further, having the ability to sense electrical activity across the target tissue while ablating may reduce the area that the clinician burns as the clinician may become aware when the procedure has been successful and cease further ablation.

In yet another embodiment, the conductive wires 39 of the first and/or second structures 20, 30 may be helically wound, as illustrated in FIG. 2. Helically winding the conductors 39 may reduce kinks forming in the shape structures 20, 30 and may reduce the strain on the structures 20, 30. As such, a helical structure may provide a more durable catheter 10 while al so maintaining sufficient flexibility.

FIG. 1 depicts an external fluid conduit 36 disposed on a portion of the second shape structure 30. Preferably, the fluid conduit 36 is disposed substantially on the outer portion of the second shape structure 30 such that when irrigation fluid is supplied to the fluid conduit 36, the fluid may be expelled through at least one fluid aperture 38. The fluid apertures may direct the irrigation fluid radially outwardly such that a ring of fluid may be energized. This may allow effective delivery of irrigation fluid to a target site, such that a uniform amount of fluid may be delivered to a target location. Optionally, the diameter of the fluid apertures 38 may vary such that a differing amount of fluid is expelled along the second shape structure 30. Varying the diameters of the fluid apertures 38 may also allow for a more uniform or targeted fluid distribution as the pressure may vary along the length of the second shape structure 30.

FIGS. 10 and 11 depict another embodiment of the second shape structure in which the fluid apertures 38 are positioned adjacent to the at least one electrode 32 at sides 33A and 33B, as seen in FIG. 1. This configuration may allow for a more even energization of irrigation fluid. It will be appreciated that the fluid conduit 36 may be in communication with one fluid aperture 38 or a plurality of fluid apertures 38. The fluid apertures 38 may preferably be formed in the arc or the wall of the fluid conduit 36 facing radially outwardly relative to the catheter sheath 12. This may allow the irrigation fluid to be disbursed toward tissue, rather than directed inwardly toward the axis of the catheter 10. However, any fluid aperture 38 configuration may be used. It will be appreciated that a fluid aperture 38 may also be an irrigation aperture 38. In at least one embodiment, the fluid conduit 36 forms an outer perimeter of the second structure 30 or at least partially faces an outer perimeter of the shape of the second structure 30.

With reference to FIG. 2, an embodiment of the second shape structure 30 is depicted. In this embodiment, the second shape structure 30 further comprises an inner tubular member 40, at least one conductor 39 around a wall of the inner tubular member 40 or embedded in the wall of the inner tubular member 40 and at least one outer tubular member 37. An external fluid conduit 36 may be integrally formed with the outer tubular member 37 or fused or otherwise fixed to the tubular member 37 such that a fluid lumen is formed between the fluid conduit 36 and the tubular member 37. The lumen formed between the outer tubular member 37 and the fluid conduit 36 may be a crescent shape, a semi-circular shape or any other predetermined shape.

In yet another embodiment (not shown), the fluid conduit 36 is disposed in the inner tubular member 40. Preferably, the fluid conduit 36 faces radially outward relative to the catheter sheath 12, such that irrigation fluid is expelled to the target site tissue and, therefore, may reduce potential ablation of non-target tissue. Further, in this configuration, irrigation fluid is generally directed away from the first shape structure 20 and, therefore, the potential electrical interference are reduced or eliminated when sensing.

At least one of the first and the second structures 20, 30 may be formed from an opaque material or a transparent material. Optionally, only the fluid conduit 36 on the second shape structure 30 is formed with a transparent material such that irrigation fluid can be viewed as it passes through the catheter ex vivo.

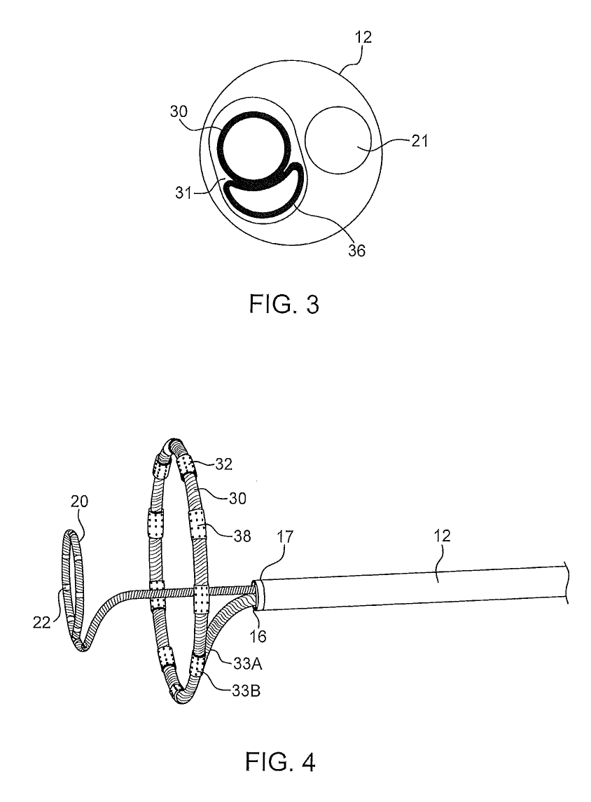

Referring to FIG. 3, there is depicted a cross-sectional view of another embodiment of the catheter sheath 12. As depicted, the catheter sheath 12 comprises a first shape lumen 21 and a second shape lumen 31. The first and second shape lumens 21, 31 are configured to receive the first and second shape structures 20, 30. It will be appreciated that more than two lumens may be formed in the catheter sheath 12.

FIG. 4 depicts an embodiment of a double shape catheter 10 of the present disclosure without an external fluid conduit 36. The fluid conduit 36 may optionally be formed within the second shape structure 30, for example, in the inner tubular member 40. If the fluid conduit 36 is formed within the second shape structure, at least one fluid aperture 38 is formed through the wiring 39 of the second shape structure. Preferably, if the fluid apertures 38 are formed through the wiring 39, the locations of the apertures 38 are formed through inert or non-conductive wiring 39 such that energization of the conductors may still be achieved. Alternatively, the apertures 38 may be formed between the conductive wiring 39.

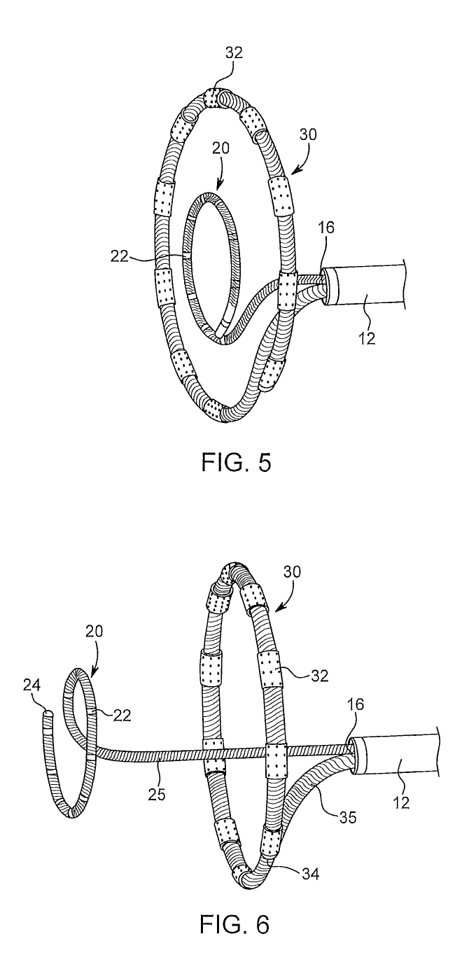

FIG. 5 illustrates a further embodiment of a double shape catheter 10 with the shape of the first shape structure 20 substantially in the same plane as that of the shape of the second shape structure 30. In this embodiment, the first shape structure 20 and the second shape structure 30 are preferably configured to be displaceable relative to each other. More preferably, the first shape structure 20 and the second shape structure 30 may be adapted to be telescopically displaceable relative to the distal end 16 of the catheter sheath 12.

Turning to FIG. 6, the first shape structure 20 is depicted as displaced distally relative to the second shape structure. It will be appreciated that the second shape structure 30 may also be displaced distally relative to the first shape structure 20. While not illustrated, at least one of the bridging portions 25, 35 of the structures 20, 30 may be adapted to deflect axially, such that the plane of the shape of one structure is either not parallel to the plane of the shape of the other structure, or the plane of one shape structure is not perpendicular to the axis of the catheter sheath 12.

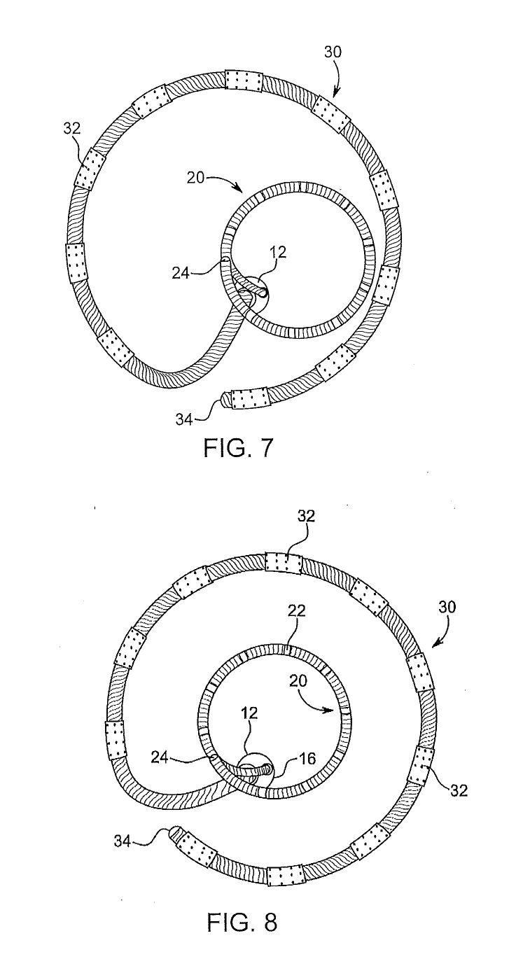

FIGS. 7 and 8 depict an embodiment of the present disclosure with the first shape structure 20 radially offset relative to the catheter sheath 12 by bridging portion 25 and the second shape structure 30 is radially offset relative to the catheter sheath 12 via bridging portion 35. Preferably, the first and/or the second shape structures 20, 30 may be rotatable about the axis of the catheter sheath 12. This may allow a clinician to more easily trek or implant the catheter along tortuous anatomy to a target tissue. Additionally, allowing the first shape structure 20 to be eccentrically disposed relative to the second shape structure 30 may allow improved positioning to perform a procedure. For example, the first shape structure 20 may be offset relative to the second shape structure 30 such that the first shape structure 20 may be wedged or in an abutting relationship with an artery or other tissue of a patient and the second shape structure 30 may be positioned closer to a target site.

As seen in FIG. 8, the shape of the first shape structure 20 and the shape of the second shape structure 30 may be concentrically aligned. While not illustrated, the axis of the catheter sheath 12 may also be concentrically configured relative to at least one of the shape of the first shape structure 20 and the shape of the second shape structure 30.

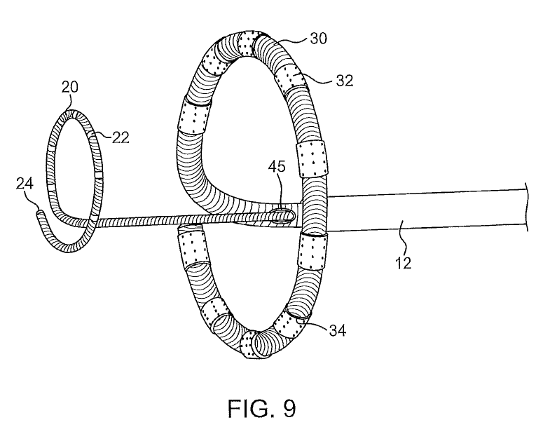

Referring now to FIG. 9, there is illustrated yet a further embodiment of a double shape catheter 10. In this embodiment, the catheter sheath 12 comprises a single lumen (not shown) through which first and second shape structures 20, 30 extend. Preferably, the second shape structure 30 comprises a structure lumen (not shown) with an aperture 45 through which the first shape structure 20 may be extended. This may reduce the overall French (Fr) diameter of the catheter sheath 12 as only one lumen is required to be formed. The aperture 45 may comprise an ingress restrictor or membrane (not shown) to prevent or restrict the ingress of fluid, such as blood, through the aperture 45 and into the structure lumen.

As illustrated in FIGS. 4 to 6, the plane of the shape of the first and second shape structures 20, 30 is generally perpendicular to the axis of the catheter sheath 12 and the shapes of the first and second shape structures 20, 30 are preferably parallel relative to one another. It will be appreciated that at least one of the first shape structure 20 and the second shape structure 30 may be configured to deflect relative to one another such that the plane of a first shape is at an angle that is not parallel to the plane of the second shape.

In yet a further embodiment, the electrodes 22, 32 may be in the same plane or flush with the respective shape structure. The term “flush” in the context of the present disclosure may mean a substantial linear relationship between two surfaces, for example, the outer surface of a shape structure 20, 30 and the respective electrodes disposed thereon, for example, as illustrated in FIG. 9. This may provide a smooth finish to the shape structures 20, 30, which may provide easier reprocessing of the shape structures 20, 30 and improve the maneuverability through tortuous anatomy.

In at least one embodiment, the clinician may guide the catheter along the tortuous anatomy of the patient using the second shape structure 30 as an introducer. After the second shape structure 30 reaches the target location, the first shape structure 20 may optionally be extended through the lumen of the second shape structure 30 to sense the target tissue.

In yet another embodiment, the catheter sheath is used as the introducer such that when the catheter is at a target location, the first and/or second shape structures 20, 30 may be inserted through the at least one lumen of the catheter sheath 12 to the target site. It will be appreciated that at least one of the first and/or second shape structures 20, 30 may be completely withdrawn from the catheter 10.

It will be appreciated that the term “loop structure,” in the context of the present disclosure, may be imparted or used and may include any predetermined shape, such as a linear shape, a non-rectilinear shape, a regular shape or an irregular shape. Altering the shape of the first and/or second shape structures 20, 30 may provide a more effective treatment option for a specialized operation or surgery.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrially applicable.

1. A double loop catheter for delivering ablation energy, the catheter comprising:
a catheter sheath having a proximal end, a distal end and at least one lumen extending therethrough;
a first loop structure having a proximal end and a distal end and a second loop structure having a proximal end and a distal end;
the first loop structure and the second loop structure receivable in the at least one lumen of the catheter sheath and being configurable to extend distally of the distal end of the catheter sheath;
the first loop structure comprising a first electrode near to the distal end of the first loop structure and the second loop structure comprising a second electrode near to the distal end of the second loop structure; and
wherein the first loop structure and the second loop structure are displaceable between a first configuration and a second configuration, wherein the first loop structure is distal relative to the second loop structure in the first configuration, and the second loop structure is distal relative to the first loop structure in the second configuration.

2. The double loop catheter of claim 1, wherein at least one of the first loop structure and the second loop structure is axially displaceable.

3. The double loop catheter of claim 1, wherein the first loop structure is adapted to be positioned in the same plane relative to the second loop structure.

4. The double loop catheter of claim 1, wherein the distal end of the catheter sheath at least restricts fluid from entering the at least one lumen of the catheter sheath.

5. The double loop catheter of claim 1, wherein the first loop structure and the second loop structure can be configured to align about a concentric axis.

6. The double loop catheter of claim 1, wherein the second loop structure comprises at least one fluid aperture.

7. The double loop catheter of claim 6, wherein the second loop structure comprises a plurality of fluid apertures.

8. The double loop catheter of claim 7, wherein the second loop structure comprises ablation electrodes, and at least one fluid aperture of the plurality of fluid apertures is disposed beside each ablation electrode, respectively.

9. The double loop catheter of claim 1, wherein at least one of the first loop structure and the second loop structure comprises a shape-imparting element adapted to impart a shape to the distal end of the respective loop structure.

10. The double loop catheter of claim 9, wherein the shape-imparting element is a stylet adapted to impart a non-rectilinear shape to the distal end of the respective loop structure.

11. The double loop catheter of claim 1, further comprising a manipulation means such that actuation of the manipulation means can alter a relative size of the loop of at least one of the first loop structure and the second loop structure.

12. The double loop structure of claim 1, wherein each electrode comprises at least one fluid aperture adapted to expel a fluid.

13. A catheter for delivering energy to a target location, wherein the catheter comprises:
a catheter sheath having a proximal end and a distal end;
a first structure having a proximal end and a distal end, and a second structure having a proximal end and a distal end;
each of the distal ends of the first and second structures having a shape imparted thereto;
each of the first structure and the second structure being adapted to extend from the distal end of the catheter sheath; and
wherein the first structure and the second structure are displaceable between a first configuration and a second configuration, and wherein the first structure is distal relative to the second structure in the first configuration, and the second structure is distal relative to the first structure in the second configuration.

14. The catheter of claim 13, wherein at least one of the first structure and the second structure comprises an electrode near to its respective distal end.

15. The catheter of claim 14, wherein the electrode is at least one of a sensing electrode and an energizing electrode.

16. The catheter of claim 13, wherein the shape is a non-rectilinear shape.

17. The catheter of claim 13, wherein at least one of the first structure and the second structure are telescopically displaceable.

18. The catheter of claim 13, wherein the second structure comprises at least one fluid aperture adapted for expelling a fluid.

19. The catheter of claim 13, wherein at least one of the first structure and the second structure comprises a shape-imparting element.

20. The catheter of claim 19, further comprising a manipulation means for manipulating the shape-imparting element.

21. The catheter of claim 13, wherein the shape of the first structure can be configured to be concentrically aligned relative to the shape of the second structure.

22. A catheter for delivering energy to a target location, comprising:
a catheter sheath having a proximal end and a distal end;
a first structure having a proximal end and a distal end, and a second structure having a proximal end and a distal end;
each of the distal ends of the first and second structures having a shape imparted thereto;
the second structure further comprising a fluid conduit disposed on at least a portion of an outer perimeter of the shape imparted thereto;
each of the first structure and the second structure being adapted to extend from the distal end of the catheter sheath; and
wherein the first structure and the second structure are displaceable between a first configuration and a second configuration, wherein the first structure is distal relative to the second structure in the first configuration, and the second structure is distal relative to the first structure in the second configuration.
